(19) United States
(12) Patent Application Publication
(10) Pub. No.: US 2006/0229608 A1
(43) Pub. Date: Oct. 12, 2006

(54) Apparatus and methods for spinal implant with dynamic stabilization system

(76) Inventors: Thomas A. Foster, Boulder, CO (US); David M. Hooper, Austin, TX (US); Margaret E. Mitchell, Cedar Park, TX (US); David J. Krueger, Cedar Park, TX (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/082,297

(22) Filed: Mar. 17, 2005

(51) Int. Cl. A61F 2/30

(52) U.S. Cl. 606/61

(57) ABSTRACT

A spinal implant provides support for desired parts of the spine. The implant can provide support in both fusion and non-fusion situations. The spinal implant includes an implant rod and fasteners for coupling or fastening the implant rod to the affected regions of the spine via the pedicles of the affected vertebrae). The implant rod includes a flexible portion and rigid end portions. The fasteners couple the end portions of the rod to the pedicles in the affected level. The flexible portion can take a variety of shapes, such as non-helical, multi-curve springs. One may combine several implant rods to provide an overall implant for more than one level of the spine. The implant can allow desired motion while tending to limit undesirable motion, thus protecting areas of the diseased or injured spine, such as the nucleus pulposus or anulus fibrosis. Furthermore, the implant can provide a combination of rigid and flexible support, as desired. One may manufacture the implant using a variety of materials, such as stainless steel, titanium, or titanium Beta C.

### TECHNICAL FIELD

The inventive concepts relate generally to spinal implants. More particularly, the invention concerns apparatus and associated methods for spinal implants with dynamic stabilization systems that can provide flexible or rigid support, as desired.

### BACKGROUND

Modern spine surgery often involves the use of spinal implants to correct or treat various spine disorders or to support the spine. Spinal implants may help, for example, to stabilize the spine, correct deformities of the spine, facilitate fusion, or treat spinal fractures. Typical spinal implants either provide flexible or rigid (i.e., in a fusion procedure) support for the affected regions of the spine. Furthermore, they either limit movement in the affected regions in virtually all directions (for example, in a fused region), or they fail to limit undesired movement of the spine while allowing the desired movement. A need exists for a spinal implant that provides flexible or rigid support, as desired, while allowing desired movement of the affected levels of the spine and limiting the undesired movement of those levels.

### SUMMARY

The inventive concepts relate to apparatus and methods for spinal implants with dynamic stabilization systems. In one exemplary embodiment, an implant includes a rod with a multi-curve flexible portion. Each end of the flexible portion couples or connects to a respective rigid portion of the rod. A pair of fasteners fasten the rod to vertebrae (e.g., at a desired level of the spine).

In another exemplary embodiment, a system for supporting vertebral bodies in a spine includes an implant rod, and a pair of fasteners. The implant rod includes a serpentine spring portion with two ends. Each of the ends of the spring portion couples or connects to a respective pair of rigid portions of the rod. Fasteners couple the implant rod to a pair of spinal vertebrae.

In yet another exemplary embodiment, a method of producing a spinal implant includes forming an implant rod from a block of material (such as titanium Beta C). The block of material is formed so as to generate a multi-curve flexible portion with two ends. The ends of the flexible portion of rod are formed so as to couple, respectively, to a pair of rigid portions of the rod.

### BRIEF DESCRIPTION OF THE DRAWINGS

The appended drawings illustrate only exemplary embodiments of the invention and therefore should not be considered or construed as limiting its scope. Persons of ordinary skill in the art who have the benefit of the description of the invention appreciate that the disclosed inventive concepts lend themselves to other equally effective embodiments. In the drawings, the same numeral designators used in more than one drawing denote the same, similar, or equivalent functionality, components, or blocks.

FIG. 1 shows an implant rod according to an illustrative embodiment of the invention.

FIG. 2 illustrates an implant rod according to another illustrative embodiment of the invention.

FIG. 3 depicts an isometric view of an implant according to an exemplary embodiment of the invention implanted to support a portion of a spine.

FIG. 4 shows a side view of the implant shown in FIG. 3.

FIGS. 5A-5C illustrate examples of implant configurations in illustrative embodiments according to the invention.

FIGS. 6A-6B depict multi-level implant rods according to exemplary embodiments of the invention.

FIG. 7 shows an implant rod according to an exemplary embodiment of the invention.

FIGS. 8A-8B illustrate an implant rod 700 according to another exemplary embodiment of the invention.

FIGS. 9A-9B depict an implant rod according to an additional exemplary embodiment of the invention.

FIGS. 10A-10B show an implant rod 900 according to another embodiment of the invention.

FIGS. 11A-11B illustrate an implant according to an exemplary embodiment of the invention implanted to support a portion of a spine.

FIGS. 12A-12C depict implant rods with orientation mechanisms according to illustrative embodiments of the invention.

### DETAILED DESCRIPTION

The disclosed novel concepts relate to spinal implants with dynamic stabilization systems. In a dynamic stabilization procedure, the surgeon typically attaches an implant construct to either side of the affected vertebral level. The implant serves the function of stabilizing the spine. The inventive implants allow a controlled range of motion in some directions (or allow some types of motion), while inhibiting or limiting motion in other directions (or inhibit or limit other types of motion). The implants allow the patient to maintain flexibility and a functioning spine. The implant can also protect the loading at a level in the early stages of degeneration and allow healing of the soft tissues and bony structures at that level.

The implants can also allow flexibility in one or more levels, while supporting fusion in one or more other levels. Fusion surgery (for example, in the case of degenerative vertebral disease), allows fusion of adjacent vertebrae. Although the fusion reduces the pain, it also limits or reduces functionality of the spine, and may affect the stresses at levels superior and/or inferior to the fused level(s). By using the appropriate combination of rigid and flexible implant rods, the surgeon can provide flexibility in one or more levels and provide fusion in one or more other levels, as desired. The implant allows selective control of the loads and ranges of motion at the levels adjacent to a fusion and may help to prevent adjacent disc disease.

The novel spinal implants provide many advantages over conventional implants, as described below in detail. The spinal implants tend to allow desired movements of the affected parts of the spine, while tending to limit undesired movement of those regions. For example, the implants allow controlled flexion or extension, which may benefit the spine and promote nutritional exchange in the disc. At the same time, the implants limit or tend to disallow torsion or shear, movements considered harmful to the affected parts of the spine, which may include the nucleus pulposus or annulus fibrosis.

The disclosed implants allow rigid or flexible support for the spine, as desired (rather than providing either rigid or flexible support exclusively). The disclosed implants also offer relative ease of manufacturing. One may manufacture implants with low-profile springs or flexible regions, with no articulating pieces (no joints) that would generate wear debris.

The implants provide a flexible mechanism for providing support for one or more levels of the spine, as desired. The surgeon may vary the type of support from one level to another. For example, for one level, the surgeon may use the implant to provide flexible support, while for another level, the implant may provide rigid support. As another example, the surgeon may use alternating flexible and rigid segments of the implant to provide the desired support for the spine.

The implants can also combine rigid and flexible rods in a modular fashion to support both fusion and non-fusion applications. The ability to custom-fit the implants to the patient's needs provides the surgeon and the patient with an improved option over conventional implants. The disclosed implants also allow preloading of the implant to create distraction or lordosis of the instrumented level(s) of the spine (i.e., help to produce a desired profile of the affected regions of the spine).

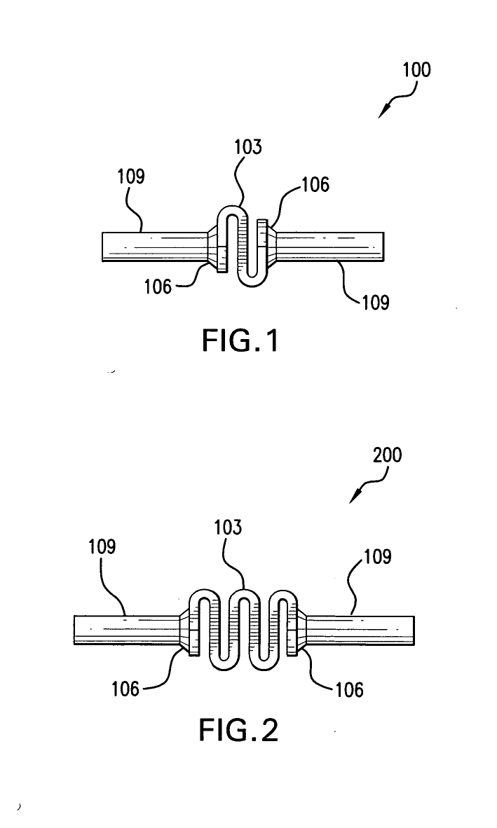

FIG. 1 shows an implant rod 100 according to an illustrative embodiment of the invention. Implant rod 100 includes a flexible or spring region (or serpentine portion or member) 103, transition portions or areas 106 (optional), and end or rigid portions 109. Spring region 103 has a multi-curve or complex shape, i.e., it includes more than one curve (for example, an “S” shape, rather than a simple “C” shape).

Spring portion 103 couples to rigid portions 109 via optional transition portions 106. Transition portions 106 serve to reduce the stress concentrations between the spring portion 103 and the rigid end portions 109. Note, however, that one may omit transition portions 106, depending on a variety of factors, such as the type of materials used, the desired strength and profile of the implant, etc., as desired. In the embodiment shown, spring portion 103 has a curvilinear, non-helical shape.

Note that spring portion 103 has two curved portions (one portion with the shape of a “U” coupled to a portion shaped like an upside-down “U”). As described below in detail, however, one may use a variety of other shapes and configurations, as desired.

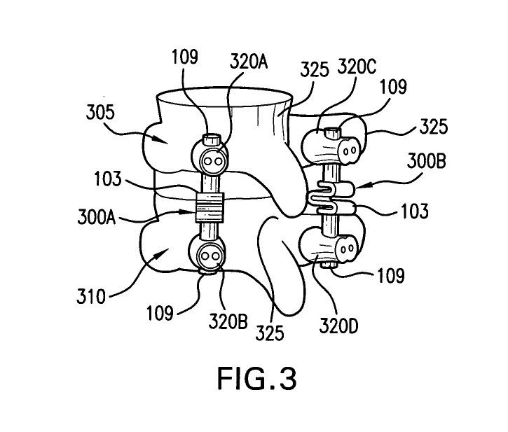

FIG. 2 illustrates an implant rod 200 according to an illustrative embodiment of the invention. Similar to implant rod 100 of FIG. 1, implant rod 200 includes spring portion 103, optional transition portions 106, and end or rigid portions 109. Spring portions 103 has a shape similar to spring portion 103 in FIG. 1.

Spring portion 103 of rod 200 includes five curved portions. As persons of ordinary skill in the art who have the benefit of the description of the invention understand, one may use a wide variety and configurations of spring portion 103, such as the number and shape of the curved regions, as desired. The number of curves and configuration of spring portion 103 depends on a number of factors, such as the patient's physical size, the materials used, the degree of flexibility desired, etc., as persons of ordinary skill in the art who have the benefit of the description of the invention understand.

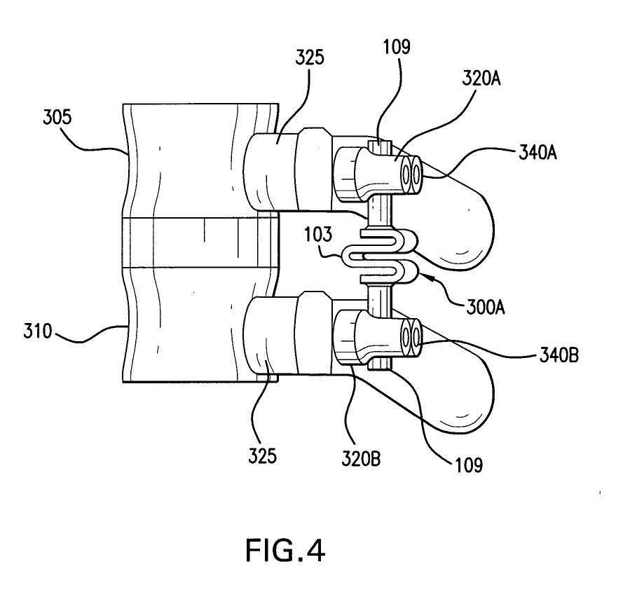

FIG. 3 shows an isometric view of an implant according to an exemplary embodiment of the invention implanted to support a portion of a spine. The figure shows vertebral body 305 and vertebral body 310. The implant uses rods 300A and 300B, one on each side of vertebral bodies 305 and 310. Implant rods, although similar to rods 100 and 200 (see FIGS. 1 and 2, respectively), differ in the number of curves of their flexible or spring portions. Note, however, that one may readily use rods with other numbers of curves or shapes, and cross-sections, as desired, and as persons of ordinary skill in the art who have the benefit of the description of the invention understand.

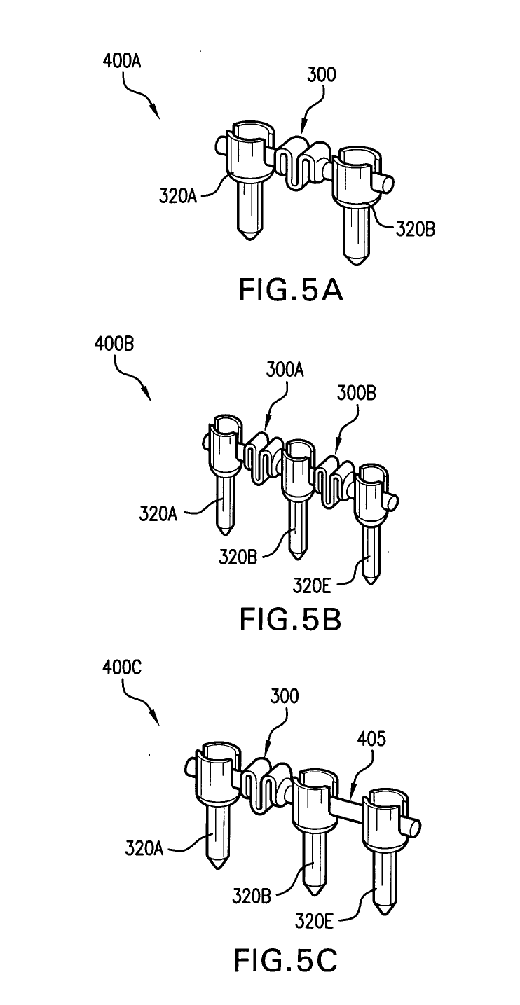

Implant rod 300A couples to vertebral bodies 305 and 310 through a desired type of fastener, such as pedicle screws 320A and 320B. More specifically, end portions 109 of implant rod 300A couple to pedicle screws 320A and 320B, respectively. Pedicle screws 320A and 320B hold end portions 109 in place so that the overall height of the implant matches the desired spacing for a particular patient. Pedicle screws 320A and 320B also fasten implant rod 300A to pedicles 325 (see FIG. 4 for location of pedicles 325) of vertebral bodies 305 and 310. Pedicle screws 320C and 320D serve similar functions with respect to implant rod 300B.

FIG. 4 illustrates a side view of the implant shown in FIG. 3. More specifically, FIG. 4 depicts implant rod 300A and pedicle screws 320A and 320B. As noted above, one may adjust the spacing between pedicle screws 320A and 320B (i.e., preload the implant) so as to achieve a desired posture for the affected portion of the spine. During the procedure, the surgeon may push towards each other (compression) pedicle screws 320A and 320B, or pull them away from each other (distraction). End portions 109 of implant rod 300A slide within the respective pedicle screws 320A and 320B. Once the surgeon has achieved the desired spacing or preloading, he or she tightens cap screws 340A and 340B. Cap screws 340A and 340B securely hold end portions of rod 300A in place and thus maintain the desired spacing between vertebral bodies 305 and 310.

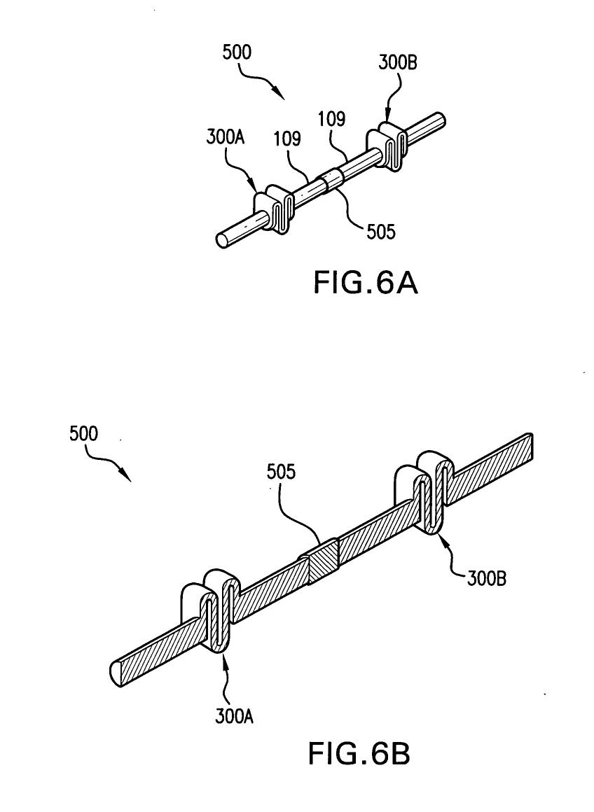

As noted above, the disclosed implants can support both fusion and non-fusion situations, in a wide variety of configurations. FIGS. 5A-5C depict examples of implant configurations in illustrative embodiments according to the invention. In FIG. 5A, implant 400A includes rod 300. Rod 300 constitutes a flexible rod, as described above. It can provide support for a non-fusion situation.

In FIG. 5B, implant 400B includes rod 300A and rod 300B. Both rod 300A and rod 300B constitute flexible rods, and can provide flexible support for two vertebral levels (flexion-flexion). Note that one may use more than two rods, as desired, and as persons of ordinary skill in the art who have the benefit of the description of the invention understand.

Referring to FIG. 5B, note that one may substitute a rigid rod for flexible rod 300A or 300B, as desired, thus providing support in a fusion situation. FIG. 5C illustrates such a configuration (flexion-rigid). More specifically, one substitutes rigid rod 405 for flexible rod 300B in order to produce implant 400C in FIG. 5C. Implant 400C provides support for a non-fusion level as well as support for a fusion level. Note, however, that by using an appropriate number and configuration of rods, one may support one or more non-fusion levels, one or more fusion levels, or both, as desired, and as persons of ordinary skill in the art who have the benefit of the description of the invention understand.

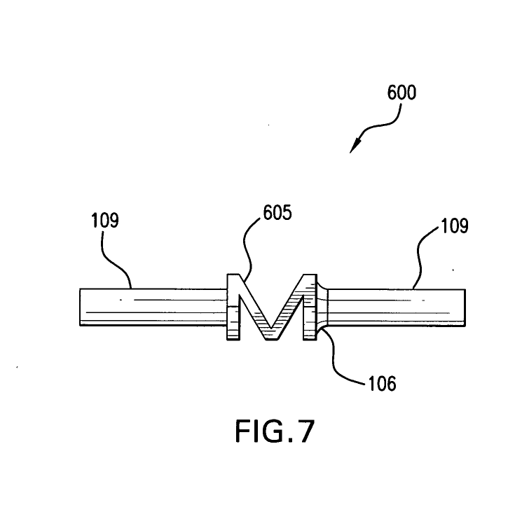

Ordinary, one may construct multi-level implant rods from one piece of material. In other words, one may process a single piece of material to produce a multi-level implant rod. Alternatively, one may construct multi-level implants by joining implant rods with a mating or coupling mechanism, as desired. FIGS. 6A-6B illustrate multi-level implant rods according to exemplary embodiments of the invention. FIG. 6A shows multi-level implant rod 500. Implant rod 500 includes rod 300A and rod 300B. Mating or coupling member 505 couples one end portion of implant rod 300A to an end portion 109 of implant rod 300B. In FIG. 6A, coupling member 505 constitutes a sleeve, although one may use other mechanisms, as persons of ordinary skill in the art who have the benefit of the description of the invention understand.

FIG. 6B shows a cut-away view of implant 500 of FIG. 6A. Coupling member 505 may have an appropriate size or length so as to allow desired spacing between rod 300A and rod 300B. By adjusting the spacing between rod 300A and rod 300B, one may fit the implant to the patient's spine or provide angulation between members 300A and 300B, as desired.

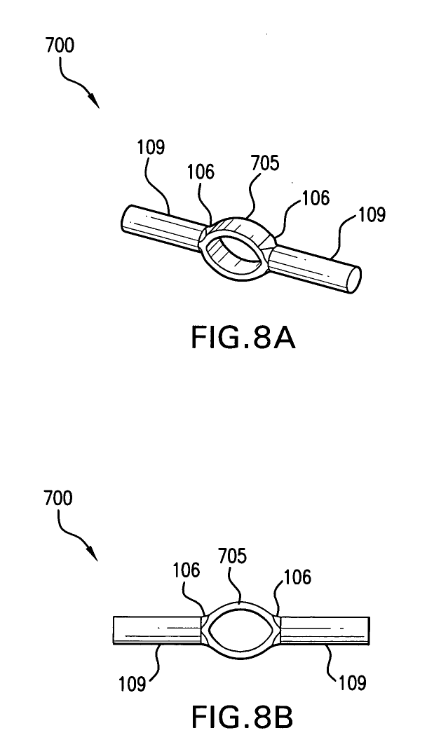

One may use a variety of configurations, shapes, and materials for the implants, including the implant rods. FIG. 7 shows an implant rod 600 according to an exemplary embodiment of the invention. Similar to the implant rods described above, implant rod 600 includes flexible portion 605, optional transition portions 106, and end or rigid portions 109. Rather than a curvilinear spring or flexible portion, spring portion 605 has an angular or substantially angular configuration. More specifically, in embodiment 600, spring portion 605 has the shape of an “M.” As in the above curvilinear designs, this angular configuration may include two or more angled sections, rather than the three angles shown.

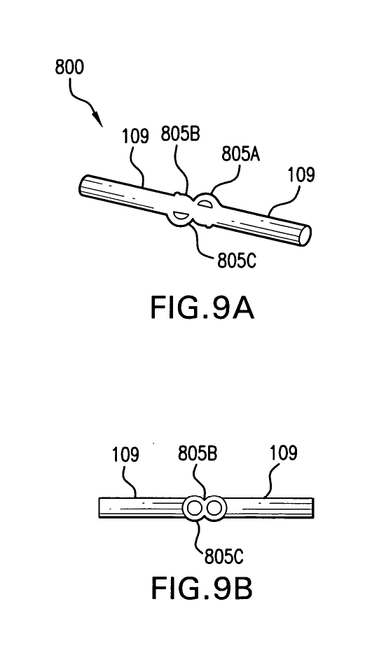

FIGS. 8A-8B illustrate an implant rod 700 according to another exemplary embodiment of the invention. FIG. 8A shows an isometric view of implant rod 700. Similar to the implant rods described above, implant rod 700 includes flexible portion 705, optional transition portions 106, and end or rigid portions 109. Flexible portion 705 has an annular, circular, or elliptical shape (e.g., a loop) of desired proportions. FIG. 8B depicts a side view of implant 700.

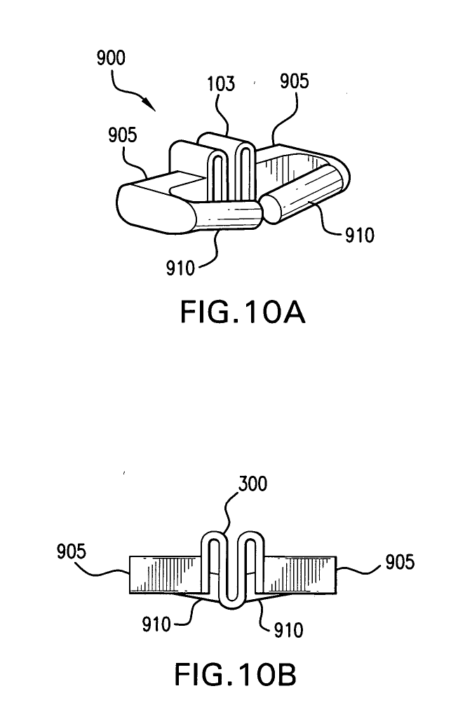

FIGS. 9A-9B illustrate an implant rod 800 according to another exemplary embodiment of the invention. FIG. 9A shows an isometric view of implant rod 800. Like the implant rods described above, implant rod 800 includes flexible portion 805, and end or rigid portions 109. Optionally, implant rod 800 may include transition portions 106, as desired. Flexible portion 805 includes one or more “S”-shaped curves of desired shapes and orientations. For example, the embodiment shown includes three “S”-shaped curves, with the two outermost curves in one orientation, and the middle curve in an opposing orientation. Alternatively, the “S” shaped curve may include three or more curved regions, rather than the two (“U” and inverted “U”). Also, the design may have two, four, or more curved sections (rather than the three curved sections shown). FIG. 9B depicts a side view of implant 800.

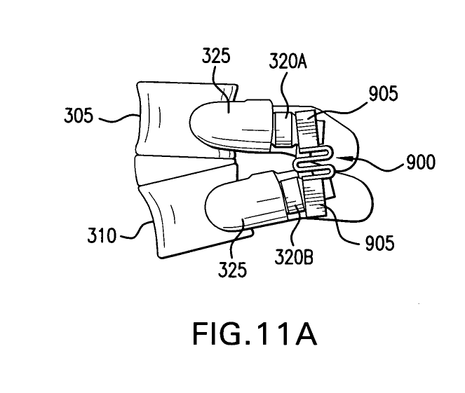

FIGS. 10A-10B illustrate an implant rod 900 according to another embodiment of the invention. FIG. 10A shows an isometric view of rod 900, whereas FIG. 10B depicts a side view.

Rod 900 includes spring or flexible portion 103, optional transition portions 905, and end or rigid portions 910. Spring portion 103 couples to rigid portions 910 via transition portions 905. Transition portions 905 provide a relatively rigid mechanism for coupling spring portion 103 to the rigid end portions 910, as desired.

In the embodiment shown, spring portion 103 has a curvilinear, non-helical shape. Note, however, that spring portion 103 may have other shapes and configurations, as desired, and as persons of ordinary skill in the art who have the benefit of the description of the invention understand. For example, spring portion 103 may have any of the shapes shown in FIGS. 7-9.

Rigid portions 910, rather than extending radially outward (see, for example, FIGS. 1-2) from transition portions 905, bend or deflect towards spring portion 103. As FIG. 10B shows, rigid portions 910 may deflect in a downward or upward direction (or a combination of the two, one for each rigid portion) from the horizontal axis of rod 900, as desired. This configuration allows the rod to be applied when the pedicle screws are closely positioned or when an offset configuration is desired. Furthermore, rigid portions 910 provide a mechanism for preloading or physically configuring the geometrical properties of implant 900.

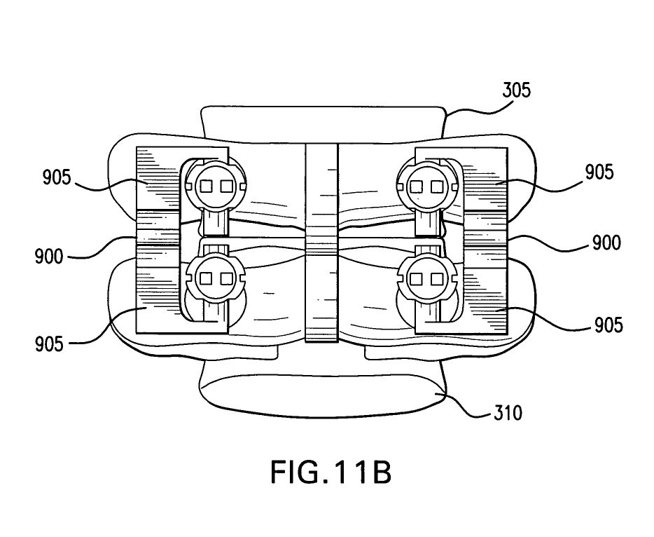

FIG. 11A shows a side view of an implant according to an exemplary embodiment of the invention implanted to support a portion of a spine. The figure shows vertebral body 305 and vertebral body 310, having pedicles 325. The implant uses rod 900, coupled to vertebral bodies 305 and 310. The implant includes another rod on the opposite side of vertebral bodies 305 and 310 (not shown in FIG. 11A).

Implant rod 900 couples to vertebral bodies 305 and 310 through pedicle screws 320A and 320B. More specifically, end portions 910 of implant rod 900 couple to pedicle screws 320A and 320B, respectively. Pedicle screws 320A and 320B hold end portions 910 in place so that the overall height of the implant matches the desired spacing and loading for a particular patient. Pedicle screws 320A and 320B also fasten implant rod 900 to pedicles 325 of vertebral bodies 305 and 310.

The surgeon can adjust the distance between the vertebral bodies 305 and 310 by preloading implant rod 900, as described above. Once the adjustment has been made, the surgeon can use pedicle screws 320A and 320B to secure rod 900, as described above. FIG. 11B shows another view of the implant in FIG. 11A. FIG. 11B shows details of the arrangement of the pedicle screws with respect to rod 900 and end portions 910.

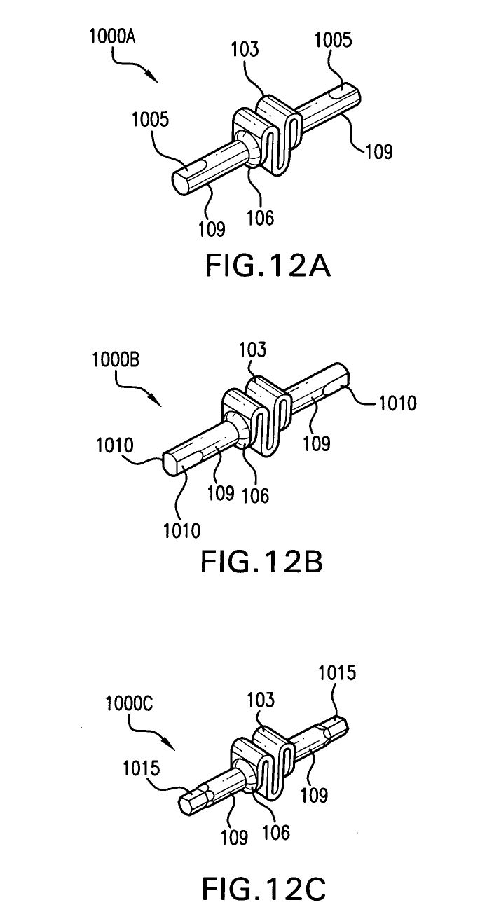

To assist in orientation and securing rods according to various embodiments of the invention, one may use a variety of orientation aids/mechanisms or location features. FIGS. 12A-12C show implant rods with orientation mechanisms according to illustrative embodiments of the invention. Persons of ordinary skill in the art who have the benefit of the description of the invention understand that one may use any of the varieties of the spring portion, described above, with the orientation and location features, as desired.

FIG. 12A shows a rod 1000A. Rod 1000A includes spring portion 103, optional transition portions 106, and end portions 109. Each end portion 109 has a flat (or substantially flat) region 1005 that allows orientation of rod 1000A (and hence spring portion 103) with respect to the affected portion of the spine.

Note that one may use a wide variety and configuration of orientation mechanisms, as desired. For example, FIG. 12B shows a rod 1000B, with spring portion 103, optional transition portions 106, and end portions 109. Each of end portions 109 includes a pair of flat (or substantially flat) regions 1010, disposed on opposing side of end portion 109.

As another example, FIG. 12C shows a rod 1000C. Rod 1000C includes spring portion 103, optional transition portions 106, and end portions 109. Each end portion 109 has a plurality of flat (or substantially flat) regions 1015 that allows orientation of rod 1000C (and hence spring portion 103) with respect to the affected portion of the spine. In rod 1000C, each of end regions 109 has six flat regions 1015, although one may use other numbers, as desired.

Note that, rather than using flat regions as described above, one may use other mechanisms, as desired, and as persons of ordinary skill in the art who have the benefit of the description of the invention understand. For example, one may use dimples, grooves, or other indicators of orientation. Furthermore, one may use various numbers of such indicators, as desired.

As noted, one may manufacture the disclosed implants (including the implant rods) from a variety of materials. For example, one may use stainless steel, titanium, other metals, or polymers, as desired. In one embodiment, one may use titanium Beta C, a titanium alloy having the composition Ti-3Al-8V-6Cr-4Mo-4Zr (or similar compositions, as desired). Titanium Beta C may be solution treated at 815° C., and aged at 565° C., or may be treated to other conditions to achieve the desired material characteristics.

Titanium Beta C provides relatively high resistance to fatigue. One may manufacture the implant rods and the pedicle screws from titanium Beta C to take advantage of that property. Using titanium Beta C helps to provide relatively robust, fatigue-resistant implants with improved longevity and performance characteristics, given the ability to vary the mechanical properties of this titanium alloy by varying the heat treat parameters

As noted above, the disclosed implants offer relative ease of manufacturing as an advantage. To manufacture the implants, one may use a lathe and wire EDM to fabricate the implant by shaping and forming a piece or block of material. Advantageously, one may manufacture each implant from a monolithic piece of material, thus reducing joints and associated manufacturing expenses. As an alternative, one may fabricate the implants by using mill processes, as desired.

Various modifications and alternative embodiments of the invention in addition to those described here will be apparent to persons of ordinary skill in the art who have the benefit of the description of the invention. Accordingly, the manner of carrying out the invention as shown and described are to be construed as illustrative only. Persons skilled in the art may make various changes in the shape, size, number, and/or arrangement of parts without departing from the scope of the invention described in this document. For example, persons skilled in the art may substitute equivalent elements for the elements illustrated and described here, or use certain features of the invention independently of the use of other features, without departing from the scope of the invention.

1. An implant, comprising:
a first rod, comprising a non-helical, multi-curve flexible portion coupled to a respective rigid portion at each end; and
a plurality of fasteners coupled to the first rod, the plurality of fasteners configured to fasten the first rod to vertebrae.

2. The implant according to claim 1, wherein the plurality of fasteners comprises a plurality of pedicle screws configured to couple the first rod to vertebrae.

3. The implant according to claim 1, wherein the first rod is preloaded before being fastened to the vertebrae.

4. The implant according to claim 1, further comprising a second rod adjacent to the first rod, wherein the second rod comprises a flexible portion.

5. The implant according to claim 1, further comprising a second rod adjacent to the first rod, wherein the second rod comprises a rigid rod.

6. The implant according to claim 1, further comprising a second rod coupled to the first rod, wherein the second rod comprises a rigid rod or a flexible rod.

7. The implant according to claim 6, further comprising a sleeve, wherein the sleeve couples the first rod to the second rod.

8. The implant according to claim 1, wherein the implant tends to limit shear and torsion movements in the spine, and wherein limiting the shear and torsion movements tends to protect the nucleus pulposus and anulus fibrosis.

9. The implant according to claim 2, wherein the rigid portions extend outwardly from the flexible portion.

10. The implant according to claim 2, wherein the rigid portions extend inwardly from the flexible portion.

11. A system for supporting vertebral bodies in a spine, comprising:
an implant rod, comprising:
a non-helical serpentine spring portion, having first and second ends; and
first and second rigid portions coupled, respectively, to the first and second ends of the spring portion.

12. The system according to claim 11, wherein the implant tends to support flexion-extension of the spine, and wherein the implant tends to limit shear and torsion movements in the spine.

13. The system according to claim 11, wherein limiting the shear and torsion movements in the spine tends to protect the nucleus pulposus and anulus fibrosis, and allows healing of the spine.

14. The system according to claim 11, further comprising first and second fasteners configured to couple the implant rod to a pair of vertebrae; wherein the first rigid portion couples to the first fastener, and wherein the second rigid portion couples to the second fastener.

15. The system according to claim 14, wherein the first fastener comprises a first screw configured to couple to a first pedicle in the spine, and wherein the second fastener comprises a second screw configured to couple to a second pedicle in the spine.

16. The system according to claim 11, wherein the first rigid portion comprises at least one orientation mechanism, and wherein each orientation mechanism is configured to facilitate orienting the spring in a desired direction.

17. The system according to claim 16, wherein each orientation mechanism comprises a flat portion.

18. The system according to claim 16, wherein the implant rod further comprises:
a first transition portion coupled to the first end of the spring portion and to the first rigid portion; and
a second transition portion coupled to the second end of the spring portion and to the second rigid portion.

19. A method of producing a spinal implant, the method comprising forming an implant rod from a block of material, wherein the rod comprises a non-helical, multi-curve flexible portion coupled to first and second rigid portions.

20. The method according to claim 18, wherein the block of material comprises titanium Beta C.

20. The method according to claim 18, wherein the spinal implant comprises a multi-level implant.

21. The implant according to claim 1 wherein the first rod comprises titanium Beta C.
